(19) United States
(12) Patent Application Publication
(10) Pub. No.: US 2024/0366481 A1
(43) Pub. Date: Nov. 7, 2024

(54) COMPOSITIONS AND METHODS FOR PANFACIAL TREATMENT OF THE DERMAL EXTRACELLULAR MATRIX

(71) Applicant: Shino Aguilera, Fort Lauderdale, FL (US)

(72) Inventor: Shino Aguilera, Fort Lauderdale, FL (US)

(21) Appl. No.: 18/653,684

(22) Filed: May 2, 2024

Related U.S. Application Data
(60) Provisional application No. 63/499,560, filed on May 2, 2023.

(51) Int. Cl. A61K 8/19; A61K 8/02; A61K 8/41; A61Q 19/08

(52) U.S. Cl. CPC A61K 8/19; A61K 8/0245; A61K 8/41; A61Q 19/08; A61K 2800/91

(57) ABSTRACT

Methods for treating the dermal extracellular matrix to counteract aging uses a composition of calcium hydroxylapatite microspheres and amino acids injected into the mesoderm. The compositions may include carboxymethylcellulose and hyaluronic acid. The compositions may also include vitamins, co-ezymes, and other nutrients. Treatment using the composition results in panfacial aesthetic improvements, reduced skin laxity, reduced wrinkle severity, reductions in hyperpigmentation, improvements in skin luminosity and subcutis thickness.

### CROSS-REFERENCE TO RELATED APPLICATIONS

This application claims priority to U.S. Provisional Application Ser. No. 63/499,560 filed on May 1, 2023.

### STATEMENT REGARDING FEDERALLY SPONSORED RESEARCH OR DEVELOPMENT

Not Applicable.

### NAMES OF PARTIES TO A JOINT RESEARCH AGREEMENT Not Applicable

### REFERENCE TO SEQUENCE LISTING, A TABLE, OR A COMPUTER PROGRAM LISTING APPENDIX SUBMITTED ON A COMPACT DISC AND INCORPORATION-BY-REFERENCE OF THE MATERIAL

Not Applicable.

### COPYRIGHT NOTICE

This disclosure is protected under United States and International Copyright Laws, All Rights Reserved. A portion of the disclosure of this patent document contains material which is subject to copyright protection. The copyright owner has no objection to the facsimile reproduction by anyone of the patent document or the patent disclosure after formal publication by the U.S. Patent Office, as it appears in the U.S. Patent and Trademark Office patent file or records, but otherwise reserves all copyright rights whatsoever.

### BACKGROUND OF THE INVENTION

### Field of the Invention

The present invention relates to compositions and methods for panfacially treating degradation of the skin's extracellular matrix which include calcium hydroxylapatite combined with amino acids and other nutrients. More particularly, the invention relates to compositions of calcium hydroxylapatite microspheres combined with a mixture of nutrients for injectable mesotherapies for treating aging skin.

### Description of the Related Art

Like many pathologies, skin aging is complex. The well-balanced, youthful architecture of young skin relies on the structure of dermis, which is like a “bed base” supporting youthful skin while fibroblasts are the “craftsmen”, producing collagen, hyaluronic acid (HA), and regulating molecules that control skin pigmentation. Dermal fibroblasts bind with extracellular matrix (ECM) fibers to produce a healthy, elastic, hydrated, and unblemished skin. Age-related alterations of the structural and mechanical support of the skin's ECM are the driving pathomechanisms of aging. Chronologically mature skin is characterized by a fragile epidermis, lying over a much thinner, and inelastic dermis, with a disoriented and defective collagen matrix support.[37] On top of that, superficial fat undergoes atrophy over time, especially in the periorbital and perioral regions.

Fibroblasts play a vital role in the skin aging process and if untreated during intrinsic or extrinsic aging, can contribute to loose, saggy skin with uneven tone. With aging, fibroblasts are reduced in numbers and their proliferative and metabolic functions slow. Simultaneously, matrix metalloproteinase concentratrons increase with aging and enzymatically contribute to the degradation of the ECM and phenotypic changes associated with aging. However, recent findings suggest that senile fibroblasts can be revived and their functions restored, particularly by restoring mechanical tension and cell-substrate contact. Targeting fibroblast activity, restoring ECM structure and mechanical tension, or mitigating degradative enzyme activity may be ideal anti-aging therapy targets.

In recent years, cosmetic dermatology has borrowed many ideas from regenerative medicine, which is loosely defined as the restoration of damaged or diseased tissues and their functions via biochemical or biomechanical cues, and has resulted in the emergence of regenerative aesthetics. Several bioregenerative treatments (i.e., calcium hydroxylapatite [CaHA]; Radiesse®, polylactic acid [PLLA; Sculptra®, polymethyl methacrylate [PMMA; Bellafill®] have been implemented in clinics and have demonstrated the ability to induce synthesis of endogenous ECM for contouring tissue in aesthetic indications. Specifically and uniquely among fillers, CaHA has demonstrated the ability to restore fibroblast function in aged or damaged skin, synthesize type I and type III collagen, elastin, and proteoglycans, and is a promising regenerative aesthetic treatment.

Since its introduction in the aesthetic market in 2006, CaHA has been an efficacious and safe injectable treatment for improving global panfacial volume loss and skin rejuvenation. CaHa injectable filler consists of 25-45 um synthetic, immunologically inert, and mechanically-stimulating microspheres suspended in an aqueous gel preparation containing carboxymethyl cellulose (CMC), glycerine, and water at a ratio of 30% microspheres and 70% gel, by volume. The calcium hydroxylapatite component provides immediate and long lasting correction of age related volume loss from bone remodeling, as well as from deep and superficial fat compartments atrophy. As the carrier gel is bioresorbed, the CaHA microspheres come into direct contact with endogenous fibroblasts and, through the activation of mechanoreceptors and restoration of mechanical tension and contact, result in sustained type I and type III neocollagenesis and elastogenesis. CaHA microspheres are eventually metabolized into calcium and phosphate ions and eliminated through the body's normal excretory pathways. Both on-label and off-label techniques utilizing CaHA have been adopted in aesthetic medicine to maximize its use as a powerful volume restorer and collagen and elastin biostimulator, ultimately contributing to ECM remodeling, youthful appearance, and optimal skin tissue mechanical properties. Of note, dilute (1:1) and hyperdilute (>1:2) CaHA formulations have demonstrated biostimulation over larger tissue volumes due to an increased microsphere spread peripheral to the injection site. Diluting CaHA allows one to adjust the mechanical and viscoelastic properties of the filler. The ability to tune the structural and biostimulatory aspects of CaHA treatments make it a favorable option in the emerging field of regenerative aesthetics and personalized medicine. Despite the significant advances in understanding mechanism of action (MOA) and biomimicry of different tissue types, clinicians are yet to consider that tissue nutritional deficits may inhibit optimal responses to biostimulatory fillers.

The role of micronutrient supplementation in wound healing and tissue regeneration has long been observed in clinical practice. Oral, intravenous, and subcutaneous administration of a variety of supplemental micronutrients has demonstrated increased wound healing rates, ECM remodeling, and collagen synthesis in a variety of pre-clinical and clinical studies. Reduced plasma concentrations of fibroblast growth factor 21 (FGF21) in Anorexia Nervosa patients has been observed, and is believed to result from malnutrition. Relatedly, fibroblast growth factor 21 has been shown to inhibit ECM degradation by autophagy flux regulation. Taken together, and without being bound by theory, the inventor hypothesized that micronutrient deficiencies may inhibit an optimal response to dermal filler-induced biostimulation via a variety of pathways.

Studies using diluted or hyperdiluted CaHa, at a 1:1 or 1:2 dilution with saline water or 2% lidocaine, respectively, have demonstrated aesthetic results, but require higher product volumes beyond recommended dosages. For example, CaHa at a 1:2 dilution results in mean total volume 5.1 ml of product. Mean recommended number of CaHA syringes is only 1.5 ml/side of the face. Furthermore, patients with poorer skin elasticity and volume loss had to be reinjected after approximately 3 months to obtain the desired aesthetic result. Recent studies combining CaHA with cross-linked HA at a dilution ratio 1:1-1:3, have demonstrated efficacy in restoring just the jaw line contour, using a mean volume of 9.61 ml. Others have mixed CaHA with HA and a variety of other diluents, none addressed skin micronutrition and all relied on higher volume injections to achieve similar results in a longer amount of time.

Mesotherapies, loosely defined as the injection or administration of a solution of amion acids, vitamins, enzymes, minerals, and other micronutrients have recently been explored in the aesthetic space for improving aging skin, though little literature has sought to elucidate which components impart significant biochemical functionality. One mesotherapy, In a series of in vitro, ex vivo, and clinical studies investigating the anti-aging effect of NCTF® 135 HA suggest that collagen I, antiglycation effect products, cell proliferation, and contractile forces were all significantly increased in vitro. Ex vivo studies demonstrated neocollagenesis, elastogenesis, improved cell proliferation, and significant tissue volumization. One clinical study demonstrated a significant increase in tonicity, hydration, and radiance, while significantly decreasing skin wrinkle score. Another clinical study demonstrated significant increases in dermal thickness and density, skin homogeneity and radiance, and significantly decreased pore size and wrinkle volume. While the empirical data across these studies demonstrate overall improvement in skin quality, the MOA is largely unknown due to the quantity of bioactive ingredients.

However, amino acids and other nutrients often interact deleteriously with known dermal fillers such as CaHa. For example, some charged and polar amino acids are known to coat CaHa microspheres, inhibiting their interactions with components of the ECM. Other amino acids have also been shown to accelerate degradation of CaHa, breaking it down into soluble ions. Some amino acids also promote crystallization, causing CaHa microspeheres to develop crystalline “spikes” which would damage and degrade the ECM.

The above-described deficiencies of today's systems are merely intended to provide an overview of some of the problems of conventional systems, and are not intended to be exhaustive. Other problems with the state of the art and corresponding benefits of some of the various non-limiting embodiments may become further apparent upon review of the following detailed description.

In view of the foregoing, it is desirable to provide compositions and methods for rejuvenating skin which contain a combination of microsphere fillers and rejuvenating mesotherapy formulations.

### BRIEF SUMMARY OF THE INVENTION

Disclosed is compositions and methods of combining CaHA, known to drive the regeneration of extracellular matrix proteins, with a blend of amino acids and other nutrients to provide the building blocks necessary to facilitate ECM regeneration, the invention supplies the metabolites required for skin regeneration directly to the treatment site, rather than relying solely on the patient's body to allocate and direct the required nutrients to the point of treatment. By providing both the catalysts and the reactants, rather than the catalysts alone, the invention consistently provides substantial improvements in panfacial aesthetics and skin luminosity, reductions in skin laxity, wrinkle severity, and hyperpigmentation, and increases skin and subcutis thicknesses following just a single treatment.

It is therefore an object of the present invention to provide formulations for improving skin by injecting into the mesoderm a combination of CaHA microspheres and a micronutrient mixture.

These and other objects and advantages of the present invention will become apparent from a reading of the attached specification and appended claims. There has thus been outlined, rather broadly, the more important features of the invention in order that the detailed description thereof that follows may be better understood, and in order that the present contribution to the art may be better appreciated. There are features of the invention that will be described hereinafter and which will form the subject matter of the claims appended hereto.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a side plan view of an injection pattern for use with a panfacial treatment of the dermal extracellular matrix in accordance with the principles of the invention;

FIG. 2 is a 5-Point GAIS graph of the results of panfacial treatment of the dermal extracellular matrix in accordance with the principles of the invention;

FIG. 3 is a 5-Point Satisfaction graph of the results of panfacial treatment of the dermal extracellular matrix in accordance with the principles of the invention;

FIG. 4 is a Merz IOH graph of the results of panfacial treatment of the dermal extracellular matrix in accordance with the principles of the invention;

FIG. 5 is a Merz Midface graph of the results of panfacial treatment of the dermal extracellular matrix in accordance with the principles of the invention;

FIG. 6 is a Merz Marionette graph of the results of panfacial treatment of the dermal extracellular matrix in accordance with the principles of the invention;

FIG. 7 is Marz Jawline graph of the results of panfacial treatment of the dermal extracellular matrix in accordance with the principles of the invention;

FIG. 8 is a Lemperle Wrinkle Severity graph of the results of panfacial treatment of the dermal extracellular matrix in accordance with the principles of the invention;

FIG. 9 is a Skin Luminosity (radiance) graph of the results of panfacial treatment of the dermal extracellular matrix in accordance with the principles of the invention;

FIG. 10 is a Subcutis Thickness graph of the results of panfacial treatment of the dermal extracellular matrix in accordance with the principles of the invention;

FIG. 11 is a Skin Thickness graph of the results of panfacial treatment of the dermal extracellular matrix in accordance with the principles of the invention;

FIG. 12 is a Change in Thickness graph of the results of panfacial treatment of the dermal extracellular matrix in accordance with the principles of the invention.

### DETAILED DESCRIPTION

The invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

The disclosed subject matter is described with reference to the drawings, wherein like reference numerals are used to refer to like elements throughout. In the following description, for purposes of explanation, numerous specific details are set forth in order to provide a thorough understanding of the various embodiments of the subject disclosure. It may be evident, however, that the disclosed subject matter may be practiced without these specific details. In other instances, well-known structures and devices may be shown in block diagram form in order to facilitate describing the various embodiments herein.

Various embodiments of the disclosure could also include permutations of the various elements as if each dependent claim was a multiple dependent claim incorporating the limitations of each of the preceding dependent claims as well as the independent claims. Unless explicitly stated otherwise, such permutations are expressly within the scope of this disclosure. Similarly, the disclosure should be interpreted as including permutations of the various elements disclosed in the Figures, unless the various elements are clearly mutually exclusive.

Unless otherwise indicated, all numbers expressing quantities of ingredients, dimensions reaction conditions and so forth used in the specification and claims are to be understood as being modified in all instances by the term “about”. The term “a” or “an” as used herein means “at least one” unless specified otherwise. In this specification and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

For ease of understanding, the following definitions will apply throughout this description; however, no definition should be regarded as being superceding any art-accepted understanding of the listed terms.

This compositions and methods of the invention provide panfacial aesthetic improvement with only a single treatment of a CaHA:NCTF HA135 blend. Treatment with the blend provided a significant improvement of at least 1-point at the MAS and Lemperle scales. The compositions of the invention are injected underneath the skin to create a reservoir of readily-available amino acids necessary for ECM protein synthesis. The proposed synergistic MOA relies upon establishing an adequate skin micronutrient profile with the NCTF 135 HA so that the protein-synthesizing effect of CaHA is not bottlenecked. The treatment methods disclosed herein provide a facial lift effect, in addition to the improvement of individual facial features, in subjects with moderate to severe mid and lower face aging. The treatment was well-tolerated, with minimal local and transient adverse reactions. The technique is a safe novel therapeutic approach, with high satisfaction rates from patients.

Regenerative aesthetics aims to restore the structure and function of aging skin, to interrupt the degradative process of chronological aging rather than simply treating symptoms. Two products, Radiesse (CaHA) and NCTF 135 HA (micronutrient cocktail) have been established as minimally-invasive treatments that restore the structure and function of parts of the skin. However, it has been anecdotally observed by the authors that some patients respond suboptimally to regenerative treatments without a clear indication as to why. Without being bound by theory, the inventor hypothesizes that micronutrient deficiencies in some patients contribute to their lack of responsiveness to certain treatments, and that a concurrent delivery of amino acids and co-enzymes create a nutritional reservoir necessary for optimal protein synthesis. Therefore, in accordance with the principles of the invention, the intervention in the aging process described herein is bimodal. Specifically, the invention comprises a bimodal method of treating, arresting, and/or reversing skin degradation. The first component, injectable CaHA, through efficient mechanotransduction of fibroblasts, synthesizes collagen and ECM components, providing mechanically robust scaffolding for new tissue formation. The second component, NCTF HA135, when injected in the skin, arms cells with the nutritional energy to function properly and produce collagen, elastin and HA. The nutritional energy provided by the components of NCTF HA135 provide a reservoir of nutrients that is immediately available to the fibroblasts recruited by the CaHA microspheres. Providing this nutrient reservoir for the fibroblast substantially and synergistically accelerates ECM regeneration.

By combining CaHA, known to drive the regeneration of extracellular matrix proteins, with a blend of amino acids and other nutrients to provide the building blocks necessary to facilitate ECM regeneration, the invention supplies the metabolites required for skin regeneration directly to the treatment site, rather than relying solely on the patient's body to allocate and direct the required nutrients to the point of treatment. By providing both the catalysts and the reactants, rather than the catalysts alone, the invention consistently provides substantial improvements in panfacial aesthetics and skin luminosity, reductions in skin laxity, wrinkle severity, and hyperpigmentation, and increases skin and subcutis thicknesses following just a single treatment.

### Materials

Radiesse® (+) Lidocaine, 1.5 cc, pre-filled syringe (Merz Pharmaceuticals GmbH, Frankfurt, Germany), was used for this study. Radiesse® (+) microspheres are biodegradable particles of CaHA, suspended in an aqueous carboxymethyl cellulose gel carrier, with 0.3% lidocaine. Radiesse® (+) has a CaHA particle size range of 25-45 micrometers. NCTF® 135 HA (FILLMED Laboratories, France) is a CE certified, class III, anti-aging and polyrevitalizing micro-filler, with a well-known efficacy and safety profile. NCTF® 135HA consists of two components: (1) 15 mg of non-cross-linked HA obtained through biotechnological processes. It is a linear polysaccharide identical to the HA present in connective tissue. (2) A polyrevitalizing solution developed to complement the HA and support its effects on the tissue. It is mainly composed of 5 groups of ingredients: vitamins, amino acids, mineral salts, coenzymes and nucleic acids. NCTF® 135HA is available in the form of the sterile and ready-to-use vials of 3 ml. NCTF® 135 HA, consists of free HA, 13 vitamins, 23 amino acids, 6 coenzymes, 6 minerals, 5 nucleic acids, and 2 reducing agents, as shown here in Table I.

TABLE I

| Class | Component |
|---|---|
| Vitamins and | Vitamin A (retinol), Vitamin C (ascorbic acid), Vitamin E |
| vitamin-like | (tocopherol), Inositol B vitamins: Vitamin B1 (thiamine), |
| substances | B2 (riboflavin), B3 (nicotinamide), B5 (pantothenic acid), |
| | B6 (pyridoxine), B8 (biotin), B9 (folic acid), B10 (P- |
| | aminobenzoic acid), B12 (cyanocobalamin) |
| Minerals | Calcium chloride, Potassium chloride, Magnesium sulfate, |
| | Sodium acetate, Sodium chloride, Sodium dihydrogen |
| | phosphate |
| Amino acids | Alanine, Arginine, Asparagine, Aspartic Acid, Cystine, |
| | Glutamine, Glutamic Acid, Glycin, Histidine, |
| | Hydroxyproline, Isoleucine, Leucine, Lysine, Methionine, |
| | Ornithine, Phenylalanine, Proline, Serine, Taurine, |
| | Threonine, Tryptophan, Tyrosine, Valine |
| Nucleosides | Deoxyadenosine, Deoxycytidine, Deoxyguanosine, |
| | Deoxythymidine, Methylcytosine |
| Co-enzymes | TPP(co-carboxylase), CoA, FAD, NAD, NADP, UTP |
| Other | Glutathione |
| antioxidants | |
| Hyaluronic | Non-reticulated sodium hyaluronate |
| acid | |

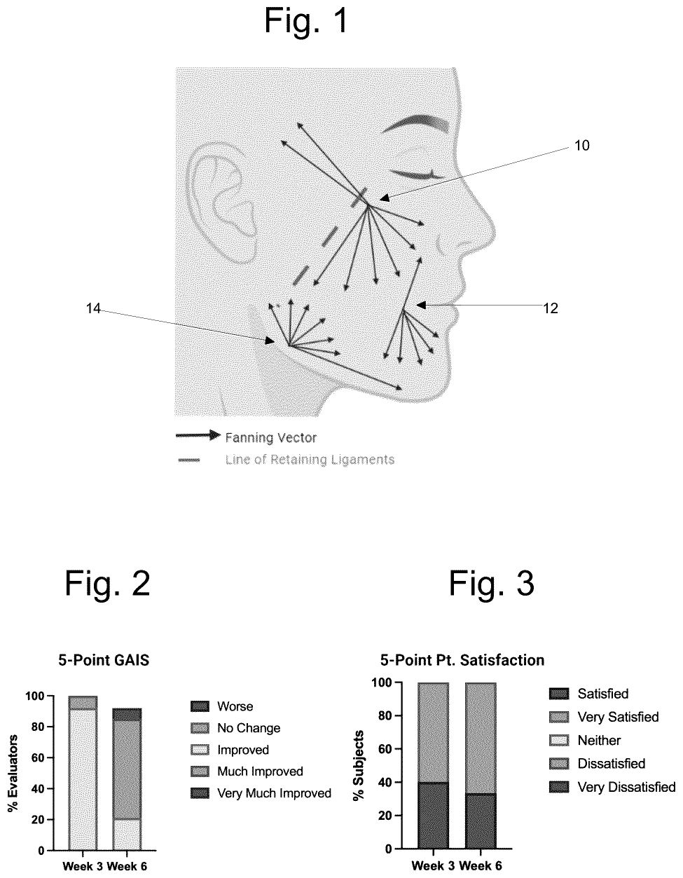

Radiesse® (+) and NCTF® 135HA were directly mixed at a 1:1 (1.5 ml of each) dilution by transferring each product into a 5 ml syringe, connecting to another syringe via a female-to-female sterile adaptor, and passing the suspension back-and-forth at least 20 times. The final suspension was a 3 ml, homogenous solution composed of 1:1 ratio Radiesse® (+): NCTF® 135 HA (FIG. 1A).

### Injection Technique

A thin layer of numbing cream, with lidocaine 2.5% and prilocaine 2.5%, was applied at the mid and lower face 30 mins prior to treatment. At the insertion points, local infiltration with 1% lidocaine with adrenaline was used. A TSK STERiGLIDE™ blunt tip cannula, 25 G, 50 mm was used by the investigators to deliver the mixture deep dermis/subcutaneously. As shown in FIG. 1, a 3-point entry approach was employed with the 1st point 10 located 1 cm in front of the zygomatic ligament, the 2nd point 12 located 1.5-2 cm from the oral commissure and the 3rd point 14 located 1.0-1.5 cm anterior to the gonial angle. Using a retrograde fanning technique, the cannula is moving from medial to lateral, placing 0.1 ml of the product in each retrograde passage. 1.5 ml of the mixture was injected on each side of the face.

### Patient Selection and Study Design

This was conducted between to evaluate the efficacy and safety of a single treatment of 1:1 CaHA:NCTF dilute mixture for facial rejuvenation and skin regeneration. Fourteen otherwise healthy individuals were recruited. The patient demographics included 12 female and 2 male patients (mean age=51.7±9.6 years, mean BMI=20.6±1.8 kg/m²). Inclusion criteria included healthy subjects over the age of 35 with a severity score at least 2 (perceptible, shallow, facial wrinkles) on the Lemperle wrinkle scale as well as a severity grade of at least 2 on the Merz Aesthetic scales for the upper and lower face. All participants were requested to avoid any cosmetic procedures in the treatment area and any excess weight gain or loss during the study. In addition, patients with active skin or systemic disease that could interfere with clinical assessment were excluded from the study. Moreover, patients with facial treatments of polylactic acid (PLLA) or HA cross-linked fillers in the past 8 months, permanent implants such as silicone, or semi-permanent fillers such as polycaptrolactone, were excluded from the study. All participants signed an informed consent prior to treatment. All subjects were asked to attend two follow up visits, one, 3 weeks post treatment (V1) and another at 6 weeks post treatment (V2). Therefore, baseline was considered the day of the treatment, V1 considered 3 weeks after, and V2 6 weeks after treatment.

### Evaluation

Multiple grading scales exist to quantify the number and depth of facial wrinkles as well as volume loss. However, there is no grading scale to directly assess the lifting effect of minimally invasive or surgical procedures of the face. Therefore, for this study, clinical efficacy was defined as the improvement of volume loss, depth and number of facial wrinkles, skin laxity and soft tissue ptosis. The lifting effect of the treatment was indirectly assessed by reduced soft tissue ptosis. Clinical efficacy was assessed by the injecting physician using the validated, photonumeric, 5-point Merz Aesthetic Scales (MAS) for infraorbital hollowness (IOH), mid face volume, marionette lines, and jawline sagging. In addition, skin quality, including facial wrinkles, was assessed using the 6-point Lemperle wrinkle severity scale. The primary endpoint was considered a 1-point improvement from baseline MAS and Lemperle scores. These patients were identified as responders. The five-point Global Aesthetic Improvement Scale (GAIS) was used for comparative ratings of aesthetic improvements, compared to baseline.

TABLE II
Summary of Scales Used and Their Grading System.

| Scale | 0 | 1 | 2 | 3 | 4 | 5 |
|---|---|---|---|---|---|---|
| Merz IOH | no | mild | moderate | severe | very severe | — |
| | hollowness | hollowness | hollowness | hollowness | hollowness | |
| Merz Upper | full upper | mildly | moderately | severely | very severely | — |
| Cheek | cheek | sunken | sunken upper | sunken | sunken | |
| (Midface) | | upper | cheek | upper cheek | upper cheek | |
| | | cheek | | | | |
| Merz | no lines | mild | moderate | severe | very severe | — |
| Marionette | | downturn | downturn | downturn | downturn | |
| Merz Jawline | no sagging | mild | moderate | severe | very severe | — |
| | | sagging | sagging | sagging | sagging | |
| Lemperle | no wrinkles | perceptible | shallow | moderately | deep wrinkle, | very deep |
| Wrinkle Scale | | just | wrinkles | deep wrinkles | well-defined edges | wrinkles, |
| | | wrinkles | | | | redundant |
| | | | | | | folds |
| 5-GAIS | — | very much | much | improved | no change | worse |
| | | improved | improved | | | |
| 5-point Patient | very | satisfied | neither | dissatisfied | very | — |
| Satisfaction | satisfied | | | | dissatisfied | |

The evaluation of clinical efficacy was conducted at baseline, V1 and V2. Adverse events were recorded by the treating investigator and categorized based on severity and relationship to the treatment. Patients were asked to score the tolerability and satisfaction of the treatment during follow up visits. Pretreatment baseline photographs of the full face were at baseline, V1, and V2. Data from 9 out of 14 patients were finally included in this evaluation process, as some patients were lost for follow-up.

Half of the patients were assessed with facial ultrasound using the portable Clarius L20 HD high frequency ultrasound scanner at 20 MHz frequency. These patients were scanned before treatment, during the baseline visit (V0), as well as post treatment at V1 and V2. The skin and subcutaneous thickness were measured at standardized points, one at the zygomatic arch, close to the nasojugal groove (A), one in the middle of the nasolabial fold (B) and one in the middle of the marionette line (C). Each parameter (skin and subcutaneous thickness) was the mean of three measurements, at each point. Then, the mean value of these measurements at each point was calculated for each parameter, for the right (R) and left (L) hemiface; e.g. Skin thickness_{R}=Mean A_{R}+B_{R}+C_{R}. The final value of each parameter was the average of measurements for the right and left hemiface; eg Skin thickness=Mean (Skin thickness_{R}+Skin thickness_{L}).

Five out of these 7 patients were assessed for skin pigmentation into a LED-UV diagnostic booth (SkinCeuticals Skinscope LED Booth, SkinCeuticals, L'Oreal Group), during V0, V1 and V2. Unwanted skin pigmentation, poor desquamation, dehydration, congested pores, and skin oiliness were reflected in a variety of fluorescent shades The presence or absence of these skin conditions was assessed as a “yes” or “no”, on the evaluation sheet.

Six of the before and after high quality pictures were selected for image analysis analyzing skin luminosity (normalized skin fluorescence). The skin luminosity of each patient was measured using a set protocol with ImageJ. Briefly, a region of interest was set to only include each patient's skin. Background image brightness was normalized to remove differences in lighting and the skin's integrated pixel density was measured and compared.

### Statistical Analysis

The Friedman's ANOVA, Kendall's coefficient and pairwise comparisons were employed to detect significant differences between groups (baseline, V1 and V2), for the non-parametric data (MAS, Lemperle and GAIS data), while the repeated measures ANOVA was used to assess differences for the parametric data (skin and subcutaneous thickness), respectively. A P value of <0.05 was considered statistically significant. The IBM SPSS software was used for the statistical analysis.

### Results

Several endpoints were investigated. In addition to the 5-point GAIS scale evaluated by the providers, patient satisfaction, regional anatomical improvements, wrinkle severity, and skin luminosity were investigated and reported herein.

There was significant improvement compared to pre-treatment at week 3 and 6 on the 5-point GAIS scale (100%, x²(1)=10.000, P<0.002, W=1,000). Notably, dramatically improved aesthetic outcomes were observed after just 3 weeks in both male and female subjects. As shown in FIG. 2, at week 3, 90% of patients were documented as improved and 10% as much improved and by week 6, 20% were much improved and 80% were very much improved. As shown in FIG. 3, all subjects were satisfied with the treatment. By week 3, 40% were satisfied, while 60% were very satisfied and by week 6, 33% were satisfied and 67% were very satisfied. All patients would recommend the treatment to somebody else. The most frequently reported AE (70%) was swelling at the injection site and all cases resolved several days post-treatment.

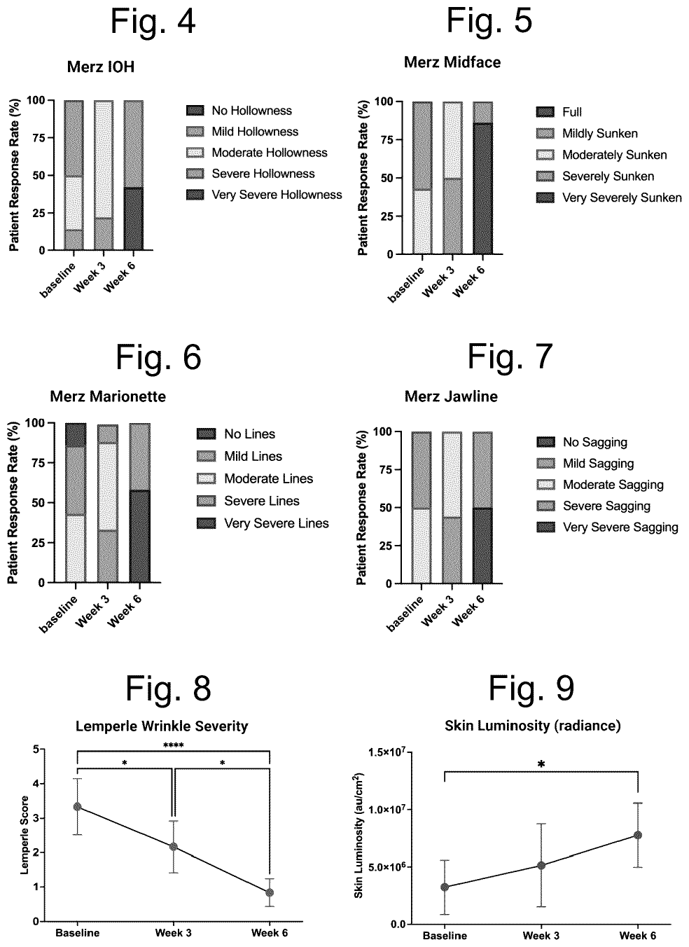

Multiple statistically significant regional anatomical improvements were observed. Referring to FIG. 4, there was a significant effect of treatment in the improvement of IOH compared to baseline (x²(2)=16.545, P=0.000, W=0.919). The mean rank score of 2.89 (severe infraorbital hollowness) at baseline improved to 2.06 (moderate hollowness) and 1.06 (mild hollowness) by weeks 3 and 6, respectively. Though there was no significant difference between baseline and week 3, significance was observed between baseline and V2 (P<0.0001) and V1 and V2 (P=0.034). There were 78% responders (with at least 1-point improvement) at V1 and 100% responders at V2.

Referring to FIG. 5, a significant effect of the treatment in the improvement of mid face volume was also observed compared to baseline (x²(2)=18.000, P=0.000, W=1.0). The mean mid face volume scores improved from 3.0 (severely sunken) at baseline to 2.0 (moderately sunken) at V1, and 1.0 (mildly sunken) at V2. Statistically significant improvements in mid face volume between baseline and V2 (P=0.000), baseline and V1 (P=0.034), and V1 and V2 (P=0.034) were observed. There were 100% responders at both V1 and V2.

Referring to FIG. 6, there was an observed significant improvement in the marionette lines compared to baseline (x²(2)=17.543, P=0.000, W=0.975). Marionette lines scores improved from 2.94 (severe lines) at baseline to 2.06 (moderate lines) at V1 and 1.0 (mild lines) at V2. Statistically significant improvement between baseline and V2 (P<0.0001) and between V1 and V2 (P=0.025) were observed. There were 89% responders at V1 and 100% responders at V2.

Referring to FIG. 7, there was a significant improvement in skin sagging and jawline contour compared to baseline (x²(2)=17.000, P=0.000, W=0.944). Mean rank scores for jawline improved from 2.94 (severe sagging) at baseline to 2.00 (moderate sagging) at V1 and 1.06 (mild sagging) at V2. Statistically significant improvements in jawlines were observed between baseline and V2 (P=0.000), baseline and V1 (P=0.045), and V1 and V2 (P=0.045). At V1, 89% of patients were responders while at V2, 100% of patients were responders.

In addition to regional improvements, skin quality was assessed with wrinkle severity and radiance. A significant improvement of wrinkles was observed in all patients. As shown in FIG. 8, a mean Lemperle Scale score of 3.0 (moderately deep wrinkles) was reduced to a mean score of 1.93 (shallow wrinkles) at week 3 and 1.07 (just perceptible wrinkles) at week 6 (x²(2)=13.556, P=0.001, W=0.968). Statistically significant improvements in wrinkle severity were observed between baseline and V2 (P=0.000) and between baseline and V1 (P=0.045). As shown in FIG. 9, skin luminosity improved from baseline at both week 3 and 6, though significance was only achieved at week 6 (N=6, P<0.05).

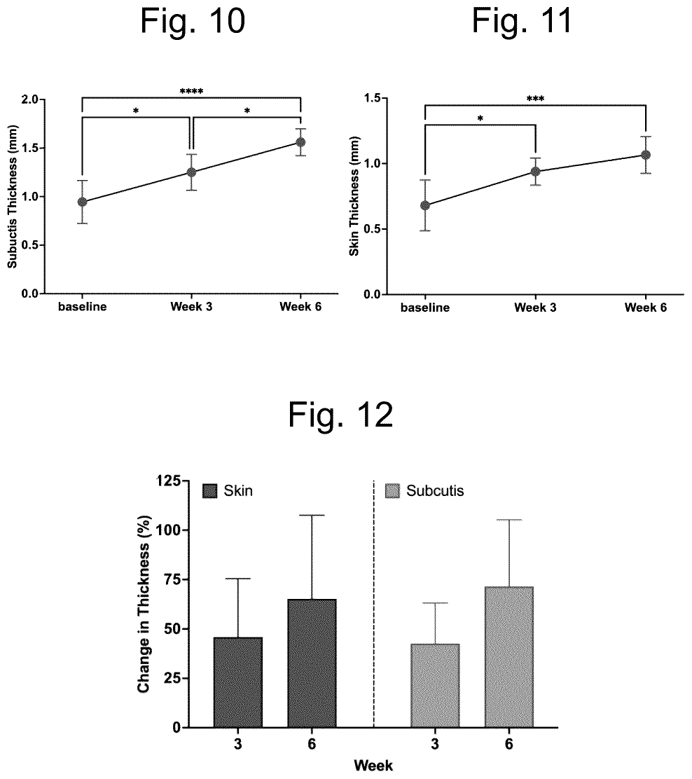

Ultrasound analysis was conducted and quantified to observe changes in skin structure and thickness. As shown in FIG. 10, the mean baseline subcutaneous thickness of 0.89±0.19 mm increased to 1.25±0.19 mm by V1 (repeated ANOVA mean difference 0.357, P=0.002) and 1.53±0.11 mm at V2 (repeated ANOVA mean difference 0.632, p=0.001). Similarly, mean baseline skin thickness was 0.68±0.21 mm at baseline and significantly increased to 0.94±0.10 mm at V1 (repeated ANOVA mean difference 0.262, P=0.019) and to 1.08±0.15 mm (repeated ANOVA mean difference 0.405, P=0.019), shown in FIG. 11. Referring to FIG. 12, skin thickness increased by 45.89%±29.60 between baseline and V1 and 65.31%±42.29 between baseline and V2 while subcutis thickness increased by 42.56%±20.52 between baseline and V1 And 71.53%±33.71% between baseline and V2.

The combination of CaHA and NCTF HA135 safely and effectively corrects many features of aging of the mid and lower face and achieves results comparable to that of surgical procedures. Skin wrinkling, laxity, and volume depletion were all restored with a single treatment in all subjects. Adverse reactions were minimal and the treatment was well tolerated by all patients. The aesthetic improvement was homogenous for all patients despite different aesthetic severities in both MAS and Lemperle scales. There were no requests by patients for touch-up treatments and all patients were satisfied at the end of the study.

Chronologically mature skin is characterized by a fragile epidermis, lying over a much thinner, and inelastic dermis, with a disoriented and defective collagen matrix support. On top of that, superficial fat undergoes atrophy over time, especially in the periorbital and perioral regions. High frequency ultrasound is an emerging imaging tool for quantifying aesthetic results of different non-surgical treatments, including mesotherapy solutions, HA fillers, biostimulators and energy devices. In line with previous ultrasonography reports on NCTF, using the compositions of the invention resulted in a significant increase in skin thickness post-treatment. Skin thickness increased by 45.89%±29.60 between baseline and week 3, and 65.31%±42.29 between baseline and week 6. Furthermore, post-treatment, there was a significant increase of the underlying subcutaneous tissue. Subcutis thickness increased by 42.56%±20.52 between baseline and week 3, and 71.53%±33.71% between baseline and week 6. The compositions and methods of the invention thus provide a significant increase in mean skin and subcutaneous thickness of the whole face, following injections of CaHA and NCTF. The assessment methods include multiple measurements from various facial points, including zygomatic, and perioral areas, thus depicting mean skin and subcutaneous thickness in a more accurate fashion.

Whereas, the present invention has been described in relation to the drawings attached hereto, other and further modifications, apart from those shown or suggested herein, may be made within the spirit and scope of this invention. Those skilled in the art will appreciate that the conception, upon which this disclosure is based, may readily be utilized as a basis for the designing of other structures, methods and systems for carrying out the several purposes of the present invention. That is, the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting. The descriptions of the embodiments shown in the drawings should not be construed as limiting or defining the ordinary and plain meanings of the terms of the claims unless such is explicitly indicated. The claims should be regarded as including such equivalent constructions insofar as they do not depart from the spirit and scope of the present invention.

1. An injectable panfacial extracellular matrix regeneration composition comprising hydroxylapatite microspheres and amino acids.

2. The injectable panfacial extracellular matrix regeneration composition of claim 1, wherein the composition is a mixture of:
1 part hydoxylapatite microspheres suspended in an aqueous carboxymethyl cellulose gel carrier, glycerine, and water at a ratio of 30% microspheres and 70% gel, by volume; and
1 part amino acid solution.

3. The injectable panfacial extracellular matrix regeneration composition of claim 2, wherein the amino acid solution comprises an aqueous hyaluronic acid solution containing amino acids, nucleosides, and co-enzymes.

4. The injectable panfacial extracellular matrix regeneration composition of claim 3, wherein the amino acid solution comprises Vitamin A (retinol), Vitamin C (ascorbic acid), Vitamin E (tocopherol), Inositol B vitamins: Vitamin B1 (thiamine), B2 (riboflavin), B3 (nicotinamide), B5 (pantothenic acid), B6 (pyridoxine), B8 (biotin), B9 (folic acid), B10 (P-aminobenzoic acid), B12 (cyanocobalamin), Calcium chloride, Potassium chloride, Magnesium sulfate, Sodium acetate, Sodium chloride, Sodium dihydrogen phosphate, Alanine, Arginine, Asparagine, Aspartic Acid, Cystine, Glutamine, Glutamic Acid, Glycin, Histidine, Hydroxyproline, Isoleucine, Leucine, Lysine, Methionine, Ornithine, Phenylalanine, Proline, Serine, Taurine, Threonine, Tryptophan, Tyrosine, Valine, Deoxyadenosine, Deoxycytidine, Deoxyguanosine, Deoxythymidine, Methylcytosine, TPP(co-carboxylase), CoA, FAD, NAD, NADP, UTP and glutathione.

5. A method for panfacial regeneration of a subdermal extracellular matrix comprising:
Providing a mixture of calcium hydroxylapatite microspheres and amino acids;
applying a thin layer of numbing cream;
selecting one or more insertion points on a region of skin to be treated; and,
subcutaneously administering the mixture to the deep dermis.

6. The method for panfacial regeneration of a subdermal extracellular matrix of claim 5 wherein the composition is a mixture of:
1 part hydoxylapatite microspheres suspended in an aqueous carboxymethyl cellulose gel carrier, glycerine, and water at a ratio of 30% microspheres and 70% gel, by volume; and
1 part amino acid solution.

7. The method for panfacial regeneration of a subdermal extracellular matrix of claim 6, wherein the amino acid solution comprises an aqueous hyaluronic acid solution containing amino acids, nucleosides, and co-enzymes.

8. The method for panfacial regeneration of a subdermal extracellular matrix of claim 7 wherein the amino acid solution comprises Vitamin A (retinol), Vitamin C (ascorbic acid), Vitamin E (tocopherol), Inositol B vitamins: Vitamin B1 (thiamine), B2 (riboflavin), B3 (nicotinamide), B5 (pantothenic acid), B6 (pyridoxine), B8 (biotin), B9 (folic acid), B10 (P-aminobenzoic acid), B12 (cyanocobalamin), Calcium chloride, Potassium chloride, Magnesium sulfate, Sodium acetate, Sodium chloride, Sodium dihydrogen phosphate, Alanine, Arginine, Asparagine, Aspartic Acid, Cystine, Glutamine, Glutamic Acid, Glycin, Histidine, Hydroxyproline, Isoleucine, Leucine, Lysine, Methionine, Ornithine, Phenylalanine, Proline, Serine, Taurine, Threonine, Tryptophan, Tyrosine, Valine, Deoxyadenosine, Deoxycytidine, Deoxyguanosine, Deoxythymidine, Methylcytosine, TPP(co-carboxylase), CoA, FAD, NAD, NADP, UTP and glutathione.

9. The method for panfacial regeneration of a subdermal extracellular matrix for panfacial regeneration of a subdermal extracellular matrix of claim 8 wherein the stop of subcutaneously administering the mixture to the deep dermis comprises using a 50 mm cannula to inject the mixture at three points, including a first point located 1 cm in front of the zygomatic ligament, a 2nd point located 1.5-2 cm from the oral commissure, and a 3rd point located 1.0-1.5 cm anterior to the gonial angle; and,
moving the cannula from medial to lateral using a retrograde fanning technique, and placing 0.1 ml of the product in each retrograde passage, with a total of 1.5 ml of the mixture was injected on each side of the face.

10. The method for panfacial regeneration of a subdermal extracellular matrix for panfacial regeneration of a subdermal extracellular matrix of claim 9 wherein the numbing cream comprises lidocaine 2.5% and prilocaine 2.5%.
